(19) United States
(12) Patent Application Publication
(10) Pub. No.: US 2006/0287301 A1
(43) Pub. Date: Dec. 21, 2006

(54) Novel formulations for phenothiazines, including fluphenazine and its derivatives

(76) Inventor: Douglas McNair, Gloucester, MA (US)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(21) Appl. No.: 11/156,016

(22) Filed: Jun. 17, 2005

(51) Int. Cl. A61K 31/5415

(52) U.S. Cl. 514/224.8

(57) ABSTRACT

The invention includes novel formulations of fluphenzine HCl, derivatives thereof and other phenothiazines for the purpose of treating a mammal, preferably a human, in need thereof.

### BACKGROUND OF THE INVENTION

Fluphenazine HCl is poorly soluble in water and other common vehicles used for the parenteral administration of drugs. Certain organic solvents may at least partially dissolve fluphenazine HCl. However, when a water-miscible organic solvent containing fluphenazine HCl at near its saturation solubility is diluted with aqueous infusion fluid, the drug tends to precipitate or adsorb to internal surfaces of the infusion set.

A generally available formulation for intramuscular injection of fluphenazine contains fluphenazine HCl at a concentration of 2.5 mg/ml and parabens as a preservative (American Pharmaceutical Partners, Inc. #28110; NDC 63323-281-10). This concentration is not ideal for cancer treatment with this drug. This is because the Cₘₐₓ that is associated with anti-cancer efficacy against multiple myeloma and other cancer cells is unsuitable. For example, when fluphenazine is used at a concentration of 2.5 mg/ml, this requires that a higher than recommended volume be used to achieve the desired Cₘₐₓ. The higher volume may in and of itself cause physiologically significant volume overload in frail patients. This is further compounded by the necessary presence of concentrated electrolyte and dextrose solutions to produce an isotonic infusate designed to avoid hemodilution that may result in cardiac arrhythmias or other complications.

Solubilization of fluphenazine HCl with surfactants facilitates dilution of saturated or near-saturated fluphenazine HCl formulations at concentrations of up to approximately 200 millimolar. However, many surfactants have serious disadvantages and, in some cases, incompatibilities with fluphenazine. For example, a Cremophor/ethanol formulation of fluphenazine HCl precipitates upon dilution with infusion fluid, and futher, fibrous precipitates form in some compositions during storage for extended periods of time. Additionally, an unexpectedly high incidence of serious hypersensitivity reactions are known to occur in some patients when Cremophor

formulations of other hydrophobic drugs have been administered. Studies have demonstrated that the Cremophor EL vehicle induces histamine release and hypotension or shock in dogs within ten minutes following intravenous administration.

Further, polyvinylchloride (PVC) infusion bags and intravenous administration sets containing drugs such as fluphenazine, usually contain diethylhexylphthalate (DEHP) as a plasticizer to maximize component flexibility. DEHP leaches to some extent into aqueous infusion fluids and blood products that come in contact with PVC materials. Exposure of animals to chronic high doses (more than 100 mg/kg) of DEHP has resulted in toxic effects including growth retardation, liver weight increase, liver damage, testicular atrophy, teratogenicity, and carcinogenicity. Addition of other solvents and surfactants may increase the amount of plasticizer leached. Therefore, there may be a substantial health risk to patients receiving fluphenazine HCl in the commercially available formulation using conventional PVC-containing equipment.

There is therefore a long felt need for improved formulations comprising fluphenazine HCl (and other phenothiazine compounds), wherein the formulations are designed to enhance stability and to minimize the clinical side effects of conventional fluphenazine HCl formulations. The present invention meets these needs.

### BRIEF SUMMARY OF THE INVENTION

The invention includes improved pharmaceutical compositions of fluphenazine HCl, derivatives thereof, and other phenothiazines, for use in treatment of diseases, disorders or conditions of the immune system in a mammal. Preferably, the mammal is a human. The pharmaceutical compositions of the invention may also include derivatives of vitamin-E derivatives, cyclodextrins, or other solubilizers in addition to specific formulations as described in more detail elsewhere herein.

The invention includes a single-use package containing the desired antineoplastic dose of fluphenazine HCl, a derivative of fluphenazine, or other phenothiazine, in an injectible volume of less than 100 ml sterile liquid.

Ideal formulations of fluphenazine, its derivatives and other phenothiazines that are suitable for pharmaceutical delivery to a mammal include formulations designed for parenteral delivery, including intravenous, intramuscular, subcutaneous and intralesional delivery. These formulations comprise sterile solutions, dispersions, emulsions, and sterile powders. The final formulation of the drug must be stable under conditions used for manufacture and storage. Furthermore, the final pharmaceutical formulation must contain protective agents to prevent contamination by microorganisms.

It should be noted that while the present disclosure exemplifies fluphenazine as the drug of choice, the invention should not be construed to be limited solely to this drug. Rather, the invention should be construed to include derivatives of fluphenazine and in addition, other phenothiazines that prove useful for treatment of diseases of the immune system.

The present invention provides new and improved formulations of fluphenazine HCl, methods of manufacturing these formulations, kits containing these formulations and methods of treating in need thereof, patients using these formulations. The new and improved formulations include pharmaceutically acceptable, water miscible solubilizers, other than Cremophor, which are believed to have improved long term stability and reduced adverse effects relative to existing formulations.

In one aspect of the present invention, a composition for delivering fluphenazine HCl in vivo is provided, which comprises fluphenazine HCl, a solvent, and a pharmaceutically-acceptable, and a water-miscible solubilizer selected from a solubilizer having the general structures set forth below:
R₁ COOR₂, R₁ CONR₂, and R₁ COR₂,
wherein R₁ is a hydrophobic C₃-C₅₀ alkane, alkene or alkyne and R₂ is a hydrophilic moiety. The solubilizer is selected such that it does not have a pKa less than about 6. Optionally, the solubilizer does not have a pKa less than about 7, more preferably not less than about 8. By designing the solubilizer so that it does not contain any acidic hydrogens, potential destabilization of fluphenazine HCl catalyzed by anionic moieties may be reduced. Upon the addition of water, the solubilizer forms micelles within which the fluphenazine HCl is solubilized in the aqueous solution.

The solubilizer may preferably be an ester (R₁ COOR₂) derived from a lipophilic acid (R₁ COOH) that has been esterified with a hydrophilic alcohol (R₂ OH). Examples of the lipophilic acids (R₁ COOH) include long chain carboxylic acids such as lauric acid, palmitic acid, stearic acid, oleic acid, linoleic acid, archidonic acid, and d-α-tocopheryl acid succinate. Examples of hydrophilic alcohols (R₂OH) include polyalcohols such as polyethylene glycols (PEG): PEG 300, 400, and 1000. In a preferred embodiment, the solubilizer is a water miscible vitamin E derivative, and is most preferably d-α-tocopherol polyethylene glycol succinate (TPGS).

The solvent in the composition should be a pharmaceutically acceptable, water miscible organic solvent that can dissolve both fluphenazine HCl (or its derivatives or other phenothiazines) and the solubilizer. Examples of suitable solvents include alcohols such as ethanol, propylene glycol and benzyl alcohol; polyalcohols such as polyethylene glycol (PEG); and amides such as 2-pyrrolidone, N-methyl-pyrrolidone and N,N-dimethyl acetamide.

The concentration of fluphenazine HCl in the composition may preferably range from about 5-20 mg/g, more preferably from about 8-15 mg/g, and more preferably from about 10-13 mg/g.

The concentration of solubilizer in the composition may preferably range from about 40 to about 90% w/w, more preferably from 45 to about 75% w/w, and most preferably from about 50 to about 60% w/w.

The weight ratio of the solubilizer to the solvent may preferably be between about 90:10 to about 40:50, more preferably between about 70:30 to about 45:55, and most preferably about 50:50.

The weight ratio of drug, e.g., fluphenazine HCl, to the solubilizer may preferably be between about 1:10 to about 1:100, more preferably about 1:20 to about 1:80, and most preferably between about 1:30 to about 1:70.

In a preferred embodiment, the composition further comprises an acidifying agent added to the composition in a proportion such that the composition has a resulting pH between about 3 and 5. The acidifying agent may be an organic acid. Examples of organic acid include ascorbic acid, citric acid, tartaric acid, lactic acid, oxalic acid, formic acid, benzene sulphonic acid, benzoic acid, maleic acid, glutamic acid, succinic acid, aspartic acid, diatrizoic acid, and acetic acid. The acidifying agent may also be an inorganic acid, such as hydrochloric acid, sulphuric acid, phosphoric acid, and nitric acid.

The composition may be diluted in an aqueous solution by adding saline or other infusion fluid for parenteral administration or intravenous injection.

In another embodiment of the invention, a composition is provided that is made as follows: combining fluphenazine HCl with a pharmaceutically-acceptable, water-miscible solubilizer as described in detail herein.

In another aspect of the present invention, a pharmaceutical formulation for delivering fluphenazine HCl in vivo is provided, which comprises water and micelles comprising fluphenazine HCl and a pharmaceutically-acceptable, water-miscible solubilizer forming the micelles, wherein the solubilizer is as described in more detail elsewhere herein.

The solubilizer contained in both the composition and the pharmaceutical formulation is an amphiphilic ester (R₁ COOR₂), an amphiphilic amide (R1 CONR₂) or an amphiphilic ketone (R₁ COR₂) which is capable of forming micelle in aqueous solution. Hydrophobic tails (R₁) of the solubilizer aggregate with lipophilic fluphenazine HCl while hydrophilic heads (R₂) of the solubilizer self-associate in water. Fluphenazine HCl is thus solubilized by associating with the hydrophobic tails of the micelles in aqueous solution.

The weight ratio of fluphenazine HCl to the solubilizer in the composition or pharmaceutical formulation may preferably be between about 1:10-1:100, more preferably about 1:20-1:80, and most preferably about 1:30-1:70.

The pharmaceutical formulation or the composition may optionally further include an excipient added to the composition in an amount sufficient to enhance the stability of the composition. Examples of the excipient includes, but are not limited to, cyclodextrin such as α-, β-, and γ-cyclodextrin and modified, amorphous cyclodextrin such as hydroxy-substituted α-, β- and γ-cyclodextrin.

Thus, there is also provided a method of making a pharmaceutical formulation, the method comprising: providing a stock compostion comprising fluphenazine HCl, a derivative thereof or other phenothiazine, a solvent and a pharmaceutically-acceptable, water-miscible solubilizer as described in more detail elsewhere herein.

One of the many advantages of the above-described pharmaceutical formulations and compositions is the use of a non-ionic, amphiphilic solubilizer for fluphenazine HCl, a derivative thereof or other phenothiazine. Previously, destabilization of e.g., fluphenazine HCl, by free carboxylate anion in formulations of Cremorphor were noted to occur. The use of an ester, an amide or a ketone reduces this destabilization. By stabilizing fluphenazine HCl in the composition, the storage shelf life for the composition can be prolonged, while the potency or pharmaceutical activity of the pharmaceutical formulation can be enhanced.

Another advantage of the pharmaceutical formulations of the present invention is that fluphenazine HCl is entrapped within the micelles formed by the solubilizer. As a result, light-induced damage to fluphenazine HCl may be reduced during the period of infusion.

A further advantage of the present pharmaceutical formulations is that the aqueous solution contains fluphenazine HCl-carrying micelles which remain physically and chemically stable. The formulation can be administered intravascularly without accompanying undue toxicity derived from undissolved drug or precipitates of the solubilizer.

In yet another aspect of the present invention there is provided a kit containing a pharmaceutical formulation for delivering fluphenazine HCl in vivo to a mammal, where the mammal is preferably a human. The pharmaceutical formulation comprises water and micelles comprising fluphenazine HCl and a pharmaceutically-acceptable, water-miscible solubilizer forming the micelles. The solubilizer is described in more detail elsewhere herein.

In yet another aspect of the present invention, a method for administering fluphenazine HCl to a host in need thereof is provided. The host is a mammal and the mammal is preferably a human.

In one embodiment of the invention, the method comprises providing a pharmaceutical formulation comprising: water and micelles comprising fluphenazine HCl and a pharmaceutically-acceptable, water-miscible solubilizer forming micelles, the solubilizer, wherein the solublizer is as set forth elsewhere herein. The pharmaceutical formulation is administered in a therapeutically effective amount to a host in need thereof. The host is a mammal, and the mammal is preferably a human.

Preferable indications that may be treated using fluphenazine HCl formulated as described in detail herein include those involving undesirable or uncontrolled cell proliferation. Such indications include restenosis (e.g. coronary, carotid, and cerebral lesions), benign tumors, a various types of cancers such as primary tumors and tumor metastasis, abnormal stimulation of endothelial cells (atherosclerosis), insults to body tissue due to surgery, abnormal wound healing, abnormal angiogenesis, diseases that produce fibrosis of tissue, repetitive motion disorders, disorders of tissues that are not highly vascularized, and proliferative responses associated with organ transplants. Treatment of a malignant disease including, but not limited to multiple myeloma, Burkitt's lymphoma, or other B-cell lymphomas is particularly contemplated in the invention.

In another embodiment, the method comprises administration to a human having any one of the diseases listed herein a pharmaceutical formulation containing fluphenazine HCl, vitamin E-TPGS (D-α-tocopheryl polyethylene glycol succinate), and solvent. According to this embodiment, fluphenazine HCl is solubilized using vitamin E-TPGS in a solvent, such as ethanol and polyethylene glycol (PEG), to form a homogenous composition. A specific, non-limiting example of vitamin E-TPGS is vitamin E-TPGS 1000 (d-α-tocopherol succinate esterified with PEG 1000).

Also according to this embodiment of the invention, the pharmaceutical formulation may further comprise an acidifying agent that is added to the formulation in an amount formulation has a resulting pH between about 3 and 5. The acidifying agent may be an organic acid including, but not limited to, ascorbic acid, citric acid, tartaric acid, lactic acid, oxalic acid, formic acid, benzene sulphonic acid, benzoic acid, maleic acid, glutamic acid, succinic acid, aspartic acid, diatrizoic acid, and acetic acid. The acidifying agent may also be an inorganic acid, including, but not limited to, hydrochloric acid, sulfiric acid, phosphoric acid, and nitric acid. The amount of acid added to the formulation may be sufficient to adjust the pH of the formulation to preferably between about pH 3 to about pH 6, more preferably between about pH 3.5 to about pH 5, and most preferably between about pH 3 to about pH 4.

The pharmaceutical formulation may optionally further include an excipient added to the composition in an amount sufficient to enhance the stability of the composition, maintain the product in solution, or prevent side effects associated with the administration of the inventive composition. Examples of excipients include but are not limited to, cyclodextrin such as α-, β-, and γ-cyclodextrin and modified, amorphous cyclodextrin such as hydroxy-substituted α-, β-, and γ-cyclodextrin. Cyclodextrins such as Encapsin™ from Janssen Pharmaceuticals or Captisol™ from CyDex may be used for this purpose.

In a particular embodiment, the method comprises administering to the patient a pharmaceutical formulation comprising fluphenazine HCl and vitamin E-TPGS at a dose of 0.1-50 mg/kg of body weight, preferably 1-20 mg/kg, more preferably 1-10 mg/kg, and most preferably 2-8 mg/kg of body weight. The administration may be repeated, preferably every two weeks, and more preferably every four weeks or several times a year.

Optionally, a desensitizer may be also administered to the patient in order to reduce any potential anaphylactic or hypersensitive responses such as allergic reactions, and or any pain associated with the administration of the pharmaceutical formulation of the invention. Examples of suitable desensitizers include, but are not limited to, steroids (such as dexamethasone, prednisone and hydrocortisone), antihistamines (such as diphenhydramine), and H-2 receptor blockers (such as cimetidine or ranitidine). The desensitizer is preferably administered to the patient prior to treatment with fluphenazine HCl formulated with vitamin E-TPGS or Captisol™.

Also optionally, a cytokine, such as, but not limited to, granulocyte-colony stimulating factor (G-CSF), may be administered (e.g., by daily subcutaneous injection) to the patient treated with fluphenazine HCl formulated with vitamin E-TPGS or Captisol™. The cytokine is administered preferably about 24 hours following treatment with fluphenazine HCl in order to ameliorate the myelosuppressive effects of antineoplastic drugs used concomitantly with fluphenazine HCl or to speed up recovery from myelotoxicity.

A wide variety of antineoplastic agents may have a therapeutic additive or synergistic effect with fluphenazine HCl formulated with vitamin E-TPGS or Captisol™. Such antineoplastic agents may be hyperplastic inhibitory agents that addictively or synergistically combine with fluphenazine HCl formulated with vitamin E-TPGS or Captisol™ to inhibit undesirable cell growth, such as inappropriate cell growth resulting in undesirable benign conditions or tumor growth. Examples of such antineoplastic agents include, but are not limited to, alkylating agents, antibiotic agents, antimetabolic agents, hormonal agents, plant-derived agents, and biologic agents.

In general, in terms of dosage, a single intravenous dose of the drug should be administerable to the mammal as a bolus infusion over several minutes. Alternatively, the formulation can be designed for use in multiple intravenous doses that are administered as bolus infusions separated in time by minutes, hours, days or weeks. Additionally, alternatively, the formulation can be designed so as to be able to administer a slow long-term infusion, or multiple short-term daily infusions, typically over the course of one to three days. Alternate day dosing, or dosing once every several days, weeks, or months, is also contemplated.

Sterile injectible solutions are prepared by incorporating the drug in the required amount in the appropriate solvent with various other ingredients as appropriate. Sterilizing procedures, such as filtration, may be used following final formulation. Typically, dispersions are made by incorporating the drug into a sterile vehicle, where in some cases, it may be useful to provide the drug in a formulation that includes micellar, cyclodextrin-complexed, or liposomal substances.

Irrespective of the components included in the final formulation of the drug, the final composition must be sterile and must be able to pass readily through an injection device such as a hollow needle. The appropriate viscosity of the final composition may be achieved and maintained using a variety of solvents and/or excipients. Prevention or inhibition of growth of microorganisms may be achieved through the addition of one or more antimicrobial agents such as chlorobutanol, ascorbic acid, parabens, or the like. It may also be preferable to include agents that alter the tonicity and ensure that the infused volume of drug into the mammal does not result in an imbalance of plasma electrolyte concentration. Agents such as sugars or salts, known to those skilled in the art, may be included to achieve the appropriate tonicity of the final composition.

Fluphenazine is marginally water soluble. Thus a preferred formulation of this compound comprises encapsulating, surrounding, or entrapping fluphenazine in, on, or by lipid vesicles or liposomes, or micelles, or cyclodextrin complexes.

Liposomes have been used successfully as formulations for administration of drugs to cancer patients. They have been shown to be useful clinically in the delivery of anticancer drugs such as doxorubicin, daunorubicin, and cisplatinum complexes (Forssen, et al., 1992, Cancer Res. 52: 3255-61; Perex-Soler, et al., 1990, Cancer Res. 50: 4260-6; and, Khokhar, et al., 1991, J. Med. Chem. 34: 325-9). Similarly, micelles have also been used to deliver medications to patients (Broden et al., 1982, Acta Pharm Suec. 19: 267-84) and micelles have been used as drug carriers and for targeted drug delivery (Lasic et al., 1992, Nature 335: 279-80; Supersaxo et al., 1991, Pharm Res. 8: 1280-1291), including cancer medications, (Fung et al., 1988, Biomater. Artif. Cells. Artif. Organs 16: 439 et seq.; Yokoyama et al., 1991, Cancer Res. 51: 3229-36).

Liposomal and/or micellar formulations containing fluphenazine, deritivatives thereof or other phenothiazines, can be synthesized using methods available to the skilled artisan and can then be administered to a cancer patient by a route also evident to the skilled artisan.

### DETAILED DESCRIPTION OF THE INVENTION

The present invention provides compositions, kits and methods for using fluphenazine HCl for treating diseases associated with abnormal B-cell or plasma cell proliferation in addition to other diseases. In particular, methods are provided for administration of fluphenazine HCl formulated with vitamin E derivative or Captisol™ to an animal, preferably a human. According to the present invention, administering to a patient fluphenazine HCl in a vehicle containing a solubilizer other than Cremophor avoids adverse effects associated with surfactants and may confer benefits in terms of stability and reduced infusion volume.

Compositions and methods that describe inhibition of the interaction of serotonin with a serotonin receptor are disclosed in U.S. Patent Application Publication No. 2003/0100570. In addition, the use of fluphenazine and derivatives thereof for modulating the immune response is described in PCT Application No. PCT/US03/19595.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

Compositions of the Present Invention

In the present invention, compositions are provided which are used for delivering fluphenazine HCl, derivatives thereof or other phenothiazines, to a mammal, preferably, a human, in vivo. While fluphenazine HCl is disclosed throughout the application as the model compound in the formulations of the invention, the invention should in no way be construed to be limited to this compound per se, because the formulations disclosed herein are equally applicable to other phenothiazines and derivatives of fluphenazine, as are the disclosed indications for these compounds.

In one embodiment of the invention, the composition comprises fluphenazine HCl, a solvent and a pharmaceutically-acceptable, water-miscible solubilizer. The solubilizer is selected from the group consisting of solubilizers having the general structures:
R₁ COOR₂, R₁ CONR₂, and R₁ COR₂,
wherein R₁ is a hydrophobic C₃-C₅₀ alkane, alkene or alkyne and R₂ is a hydrophilic moiety, the solubilizer being selected such that it does not have a pKa less than about 6. Upon the addition of water, the solubilizer forms micelles within which the fluphenazine HCl is solubilized in the aqueous solution.

The composition comprising fluphenazine HCl is formulated based on a combination of a non-ionic, amphiphilic solubilizer that forms micelles to solubilize fluphenazine HCl in an aqueous solution, and a solvent that can dissolve fluphenazine HCl and disperse the solubilizer in the composition to form a homogenous composition.

A pharmaceutical formulation can be formed comprising the composition by adding an aqueous solution such as water, saline or other infusion fluid to the composition. When an aqueous solution is added, hydrophobic tails of the solubilizer aggregate with fluphenazine HCl and entrap fluphenazine HCl within a micelle, thereby solubilizing and stabilizing fluphenazine HCl in the resultant pharmaceutical formulation.

In the composition, the solubilizer is an ester, an amide or a ketone with a pKa less than about 6. As a result, the solubilizer is essentially non-ionic under pH 6 in an aqueous solution. Optionally, the solubilizer is selected such that the solubilizer does not have a pKa less than about 7, and more preferably not less than about 8. Maintaining non-ionicity of the solubilzer is believed to prevent destabilization of fluphenazine HCl catalyzed by anions such as carboxylate. The present invention employs an amphiphilic ester as the solubilizer in the composition. Thus, carboxylate anion-catalyzed decomposition of fluphenazine HCl may be minimized, thereby enhancing the stability and prolonging storage shelf-life of the drug.

The solubilizer R₁ COOR₂ may preferably be an ester derived from lipophilic acids (R₁ COOH) that are esterified with hydrophilic alcohol (R₂ OH). Examples of lipophilic acids (R₁ COOH) include long chain carboxylic acids such as lauric acid, palmitic acid, stearic acid, oleic acid, linoleic acid, arachidonic acid, and d-α-tocopheryl acid succinate. Examples of hydrophilic alcohols (R₂ OH) include polyalcohols such as polyethylene glycols (PEG): PEG 300, 400, and 1000. In a preferred embodiment, the solubilizer is a water miscible vitamin E derivative, and most preferably is d-α-tocopherol polyethylene glycol succinate (TPGS).

The solvent in the composition comprising fluphenazine HCl is preferably a pharmaceutically acceptable, water miscible, nonaqueous solvent that can dissolve both fluphenazine HCl and the solubilizer. In the context of this invention, these solvents should be construed to include solvents that are generally acceptable for pharmaceutical use, i.e., they should be substantially water-miscible, and substantially non-aqueous. Preferably, these solvents do not cause phthalate plasticizes to leach when the solvents are used with medical equipment whose tubing contains phthalate plasticizers. Preferred examples of the pharmaceutically-acceptable, water-miscible, non-aqueous solvents that may be used in the invention include, but are not limited to, N-methylpyrrolidone (NMP); propylene glycol; polyethylene glycol (e.g. PEG300, PEG400, PEG1000); ethyl acetate; dimethyl sulfoxide; dimethyl acetamide; benzyl alcohol; 2-pyrrolidone; benzyl benzoate; C₂₋₆ alkanols; 2-ethoxyethanol; alkyl esters such as 2-ethoxyethyl acetate, methyl acetate, ethyl acetate, ethylene glycol diethyl ether, or ethylene glycol dimethyl ether; (s)-(−)-ethyl lactate; acetone; glycerol; alkyl ketones such as methylethyl ketone or dimethyl sulfone; tetrahydrofuran; cyclic alkyl amides such as caprolactam; decylmethylsulfoxide; oleic acid; aromatic amines such as N,N-diethyl-m-toluamide; or 1-dodecylazacycloheptan-2-one.

Most preferred examples of pharmaceutically-acceptable, water-miscible, non-aqueous solvents include alcohols such as ethanol, propylene glycol and benzyl alcohol; polyalcohols such as polyethylene glycol (PEG 300, PEG 400, etc.); and amides such as 2-pyrrolidone, N-methyl-pyrrolidone and N,N-dimethyl acetamide. Additionally, triacetin may also be used as a pharmaceutically-acceptable, water-miscible, non-aqueous solvent, as well as functioning as a solubilizer in certain circumstances.

Pharmaceutical grade fluphenazine HCl suitable for use in this invention may be obtained from a variety of sources, including American Pharmaceutical Partners (Schaumburg, Ill.). In the context of this invention, fluphenazine HCl is intended to include fluphenazine HCl proper, and fluphenazine HCl derivatives, analogs, metabolites, and prodrugs thereof. Pharmaceutical grade fluphenazine decanoate may be obtained from Bedford Labs (Bedford, Ohio). It is also noted herein that fluphenazine is also known in the art by the names Prolixin™ and Permitil™.

The composition of the invention may contain varying amounts of each of the fluphenazine HCl, a pharmaceutically-acceptable, water-miscible solubilizer, solvent, and other excipients. In a preferred embodiment, the composition comprises fluphenazine HCl in an amount ranging from about 5-50 mg/g, more preferably from about 8-35 mg/g, and most preferably from about 10-15 mg/g.

In another preferred embodiment, the composition comprises a solubilizer in an amount ranging from about 40 to about 90% w/w, more preferably from about 45 to about 75% w/w, and most preferably from about 50 to about 60% w/w.

In yet another preferred embodiment, the weight ratio of the solubilizer to the solvent may be between about 90:10 to about 40:50, more preferably between about 70:30 to about 45:55, and most preferably about 50:50.

In another preferred embodiment, the weight ratio of fluphenazine HCl to the solubilizer may be between about 1:5 to about 1:100, more preferably about 1:10 to about 1:80, and most preferably between about 1:15 to about 1:70.

In yet another preferred embodiment, the composition further comprises an acidifying agent added to the composition in a proportion such that the composition has a resulting pH between about 3 and 5. Adding an acidifying agent to the composition serves to further stabilize the bond to the carbonyl bond of the solubilizer and prevent carbonyl anion-catalyzed decomposition of fluphenazine HCl.

Optionally, the solubilizer does not have a pKa less than about 6 or 7, more preferably not less than about 8. By designing the solubilizer not to include a proton donor under physiological conditions, potential destabilization of fluphenazine HCl catalyzed by anionic moieties may be reduced.

The acidifying agent may be an organic acid including, but not limited to, ascorbic acid, citric acid, tartaric acid, lactic acid, oxalic acid, formic acid, benzene sulphonic acid, benzoic acid, maleic acid, glutamic acid, succinic acid, aspartic acid, diatrizoic acid, and acetic acid. The acidifying agent may also be an inorganic acid, including, but not limited to, hydrochloric acid, sulphuric acid, phosphoric acid, and nitric acid. The amount of acid added to the composition may be sufficient to adjust the pH of the composition at preferably between about pH 3-6.

The pharmaceutical formulation or the composition may optionally further include an excipient added to the composition in an amount sufficient to enhance the stability of the composition, maintain the product in solution, or prevent side effects associated with the administration of the inventive composition. Examples of excipients include but are not limited to, cyclodextrin such as α-, β-, and γ-cyclodextrin and modified, amorphous cyclodextrin such as hydroxy-substituted (-., (-, and (-cyclodextrin. Cyclodextrins such as Encapsin™ from Janssen Pharmaceuticals or a β-cyclodextrin sulfobutyl ether, such as Captisol™ from CyDex (www.cyclexinc.com/captisol.html). Alternatively, the composition may also be diluted into an aqueous solution to form a pharmaceutical formulation by adding saline or other infusion fluid for parenteral administration or intravenous injection.

Pharmaceutical Formulations of the Present Invention

In the present invention, pharmaceutical formulations for delivering fluphenazine HCl to a mammal, preferably a human, in vivo are also provided. Such formulations comprise water and micelles comprising fluphenazine HCl and a pharmaceutically-acceptable, water-miscible solubilizer forming the micelles, the solubilizer selected from the group consisting of solubilizers having the general structures:
R1 COOR2, R1 CONR2, and R1 COR2,

wherein R1 is a hydrophobic C3-C50 alkane, alkene or alkyne and R2 is a hydrophilic moiety, the solubilizer being selected such that it does not have a pKa less than about 6.

The pharmaceutical formulation can be used for delivering fluphenazine HCl in vivo to a mammal, preferably via parenteral administration. Parenteral administration is the preferred approach for fluphenazine HCl as a therapy for systemic malignancies including multiple myeloma. The formulation of the present invention contains a non-ionic ester solubilizer which forms micelles in aqueous solution to solubilize fluphenazine HCl without causing precipitation, and delivers the drug into venous circulation of the body.

Generally, micelles can solubilize otherwise insoluble organic material by incorporating the organic material within their hydrophobic interior. The micelle in a pharmaceutical formulation is an association colloid that displays regions of decreasing water solubility going from the outside of the structure to the inside. Micelles are formed by amphiphilic molecules with both hydrophobic and hydrophilic moieties. In the present invention, the solubilizer is an amphiphilic ester with a hydrophobic tail (R1) and a hydrophilic head (R2). The hydrophobic tail of the solubilizer aggregates with lipophilic fluphenazine HCl to form the interior of the micelle while the hydrophilic head (R2) of the solubilizer self-associates with other hydrophilic heads and faces water outside of the micelle. Fluphenazine HCl which is substantially insoluble in aqueous solution is thus solubilized by micelle formation.

The micelles may preferably be non-ionic, such that the head group region of a micelle resembles a concentrated aqueous solution of solute. A non-ionic head group, e.g. sugar or PEG, becomes hydrated by the aqueous solution and solubilizes the micelle. The non-ionic tail group, e.g. long hydrocarbon chain, aggregates with the lipophilic drug via van der Waals interactions, and occupies a range of areas by changing its extended length, compressing or extending its hydrocarbon chain.

The solubilizer (R1 COOR2) may preferably be an ester derived from lipophilic acids (R1 COOH) that are esterified with hydrophilic alcohol (R2 OH). Examples of the lipophilic acids (R1 COOH) include long chain carboxylic acids such as lauric acid, palmitic acid, stearic acid, oleic acid, linoleic acid, arachidonic acid, and d-(-tocopheryl acid succinate. Examples of hydrophilic alcohols (R2 OH) include polyalcohols such as polyethylene glycols (PEG): PEG 300, 400, and 1000. In a preferred embodiment, the solubilizer is a water miscible vitamin E derivative, and most preferably is d-(-tocopherol polyethylene glycol succinate (TPGS).

TPGS is derived from vitamin E by esterification of the acid group of d-(-tocopherol succinate with polyethylene glycol. In particular, the commercially available TPGS 1000 esterified with PEG 1000 (Eastman Chemical Company) is water soluble up to approximately 20% w/w and stable under heat sterilization conditions. In addition, the viscosity of TPGS 1000 appears to be constant and low at concentrations below 20% w/w, a desirable property for a pharmaceutical formulation used in parenteral administration.

Other water miscible, amphiphilic solubilizer derived from d- or dl-(-tocopherol may also be used. For example, d- or dl-(-tocopherol may be esterified by water soluble aliphatic dicarboxylic acid such as malonic, succinic, glutaric, adipic, pimelic and maleic acid to form a salt, which is then further esterified with hydrophiles such as PEG to produce water miscible, amphiphilic solubilizers.

In another preferred embodiment, the weight ratio of fluphenazine HCl to the solubilizer may be between about 1:5-1:100, more preferably about 1:10-1:80, and most preferably about 1:15-1:70.

The pharmaceutical formulation can be used for delivering fluphenazine HCl to a mammal, preferably a human, in vivo, preferably via parenteral or intravenous administration. Since the aqueous formulation contains fluphenazine HCl-carrying micelles which remain physically and chemically stable, this formulation can be administered intravascularly without undue toxicity from undissolved drug or precipitates of the solubilizer and still maintains its pharmacological potency. Further, in this formulation, fluphenazine HCl is entrapped within the micelles formed by the solubilizer, thus light-induced damage to fluphenazine HCl may be reduced during the period of infusion.

In a particular embodiment, the fluphenazine HCl formulation comprises a derivative of vitamin E, vitamin E-TPGS (D-(-tocopheryl polyethylene glycol succinate). In addition, the formulation contains a solvent that can dissolve fluphenazine HCl and disperse vitamin E-TPGS to form a homogenous composition, such as ethanol and polyethylene glycol (PEG).

It is believed that upon the addition of water, vitamin E-TPGS forms micelles within which the fluphenazine HCl is solubilized in the aqueous solution. Prior to administration, for example intravenous (I.V.) infusion, fluphenazine HCl formulated with vitamin E-TPGS or Captisol™ can be dispersed or diluted with water, saline or other infusion fluid. When an aqueous solution is added, hydrophobic tails of vitamin E-TPGS (the vitamin E moiety) aggregate with fluphenazine HCl and entrap fluphenazine HCl within a micelle, thereby solubilizing and stabilizing fluphenazine HCl in the resultant pharmaceutical formulation.

The fluphenazine HCl formulated with vitamin E-TPGS or Captisol™ can be used for delivering fluphenazine HCl in vivo to a mammal, preferably a human. Delivery is preferably via parenteral or intravenous administration. Since the aqueous formulation contains fluphenazine HCl-carrying vitamin E-TPGS micelles which remain physically and chemically stable, this formulation can be administered intravascularly without undue toxicity from undissolved drug or precipitates of the solubilizer and still maintain its pharmacological potency. Further, in this formulation, fluphenazine HCl is entrapped within the micelles formed by vitamin E-TPGS, thus light-induced damage to fluphenazine HCl may be reduced during the period of infusion.

In addition, vitamin E-TPGS is essentially non-ionic under pH 6 in an aqueous solution. Maintaining non-ionicity of the solubilzer is believed to prevent destabilization of fluphenazine HCl catalyzed by anions such as carboxylate. By comparison, the commercially available fluphenazine HCl formulation having 50:50 ethanol: Cremophor, contains carboxylate moieties which ionize and may contribute to the decomposition of fluphenazine HCl in the formulation. Thus, by employing an amphiphilic ester as the solubilizer in the composition, carboxylate anion-catalyzed decomposition of fluphenazine HCl may be minimized, thereby enhancing the stability and prolonging storage shelf-life of the drug.

Methods of Making Pharmaceutical Compositions

The present invention also provides a method of making the pharmaceutical compositions of the invention. In one embodiment, a pharmaceutical composition is made by providing fluphenazine HCl, and combining it with a pharmaceutically-acceptable, water-miscible solvent and a pharmaceutically-acceptable, water-miscible solubilizer. Wherein the solubilizer is as set forth elsewhere herein.

In one variation of the embodiment, the pharmaceutical composition is prepared by dissolving fluphenazine HCl in a small quantity of a pharmaceutically-acceptable, water-miscible solvent with moderate agitation. The required volume of the pharmaceutical composition is then made up using the solubilizer dissolved in the solvent and the other components of the composition which are then mixed thoroughly.

When the pharmaceutical composition further comprises at least one excipient, the excipient, for example, hydroxypropyl cyclodextrin, is also dissolved in an aliquot of the pharmaceutically-acceptable, water-miscible solvent. This solution is then combined with a premixed solution of fluphenazine HCl and solubilizer as described herein. Any remaining volume is made up using the solvent.

When the pharmaceutical composition further comprises an acidifying agent, the acidifying agent, is added to the premixed solution of fluphenazine HCl and solubilizer as described herein and mixed under moderate agitation. Examples of an acidifying agent include organic acids such as ascorbic acid, citric acid, tartaric acid, lactic acid, oxalic acid, formic acid, benzene sulphonic acid, benzoic acid, maleic acid, glutamic acid, succinic acid, aspartic acid, diatrizoic acid, and acetic acid, and inorganic acids, such as hydrochloric acid, sulphuric acid, phosphoric acid, and nitric acid. The amount of the acidifying agent added is sufficient to adjust the pH of the final formulation to the desired range after dilution of the pharmaceutical composition with an infusion fluid, for example, saline.

In another embodiment, the pharmaceutical composition is made by providing a compostion comprising fluphenazine HCl, a solvent and a pharmaceutically-acceptable, water-miscible solubilizer as described elsewhere herein. The composition is then combined with an aqueous solution, wherein, upon addition of the aqueous solution, the solubilizer forms micelles within which the fluphenazine HCl is solubilized in the aqueous solution.

A kit containing a pharmaceutical formulation for delivering fluphenazine HCl in vivo is also provided, wherein the kit contains the components of the composition as described herein and instructions for using the kit are also provided.

Methods of Administration of the Fluphenazine HCl Formulations of the Invention

The invention further includes a method of administering fluphenazine HCl to a mammal, preferably a human. The method comprises providing the pharmaceutical composition of the invention and administering the composition to the mammal in a therapeutically effective amount.

The pharmaceutical formulation of the invention is administered to the mammal in any medically suitable manner, preferably parenterally, more preferably intravenously. The pharmaceutical formulation is prepared for administration by diluting the composition in sterile water, normal saline, D5W, Ringer's solution or other equivalent infusion liquids. Dilution of the pharmaceutical composition is preferably in the range from about 5:1 to about 1:10 v/v of the composition to the diluting solution. The extent of the dilution may be adjusted according to specific treatment schemes adopted by clinicians. The ratio of v/v in this context refers to the ratio of the volume of the composition before dilution with the infusion fluids to the total volume of the pharmaceutical formulation following dilution with the infusion fluid. Additionally, the pharmaceutical composition may be administered to the mammal in as a bolus injection.

When administering therapeutic agents such as fluphenazine HCl, a highly stable formulation is desirable. Chemical stability of a formulation generally refers to the amount of chemical degradation of a particular agent in the formulation. Chemical stability of a pharmaceutical formulation depends upon the amount of chemical degradation of the active pharmaceutical ingredient in that preparation. Commonly, stability analysis of a pharmaceutical preparation, such as a liquid parenteral product, may be performed under accelerated temperature conditions, such as in a 50° C. oven. Acceptable stability is well understood by one of skill to mean chemical stability that is sufficient for the material to be well accepted in clinical use, that definition being used herein. In a preferred embodiment, the chemical stability of fluphenazine HCl in a 50° C. oven over four weeks is greater than about 85%. In a more preferred embodiment, the chemical stability of fluphenazine HCl in a 50° C. oven over four weeks is greater than about 90%. In a yet more preferred embodiment, the chemical stability of fluphenazine HCl in a 50° C. oven over four weeks is greater than about 93%. In a most preferred embodiment, the chemical stability of fluphenazine HCl in a 50° C. oven over four weeks is greater than about 96%. As described in more detail in the Examples section, the chemical stability of fluphenazine HCl formulated with vitamin E-TPGS or Captisol™ in a 50° C. oven over four weeks is greater than 95% (Tables 2 and 5). In addition, fluphenazine HCl at 12.5 mg/g in 50:50 ethanol: vitamin E-TPGS or fluphenazine HCl at 75 mg/g in or Captisol™ did not cause precipitation within 24 hours of dilution with normal saline (Tables 3 and 6).

The fluphenazine HCl formulations as described herein are used for delivering fluphenazine HCl in vivo via various routes of administration. For example, the formulation may be administered or coadministered with other therapeutic agent(s) parenterally, intraperitoneally, intravenously, intraarterially, intramuscularly, via local delivery (for example by catheter or stent), or intrathecally.

Parenteral administration has been the preferred approach for fluphenazine HCl as a therapy for systemic malignancies. Unfortunately, the currently available fluphenazine HCl formulation which is based on a combination of ethanol and polyoxyethylated castor oil (Cremophor.RTM., BASF, Germany) can precipitate when added to an infusion fluid. Cremophor has also been associated with a series of clinical side effects necessitating extensive premedication to desensitize the individual receiving therapy. Fluphenazine HCl formulated in a non-ionic ester solubilizer such as vitamin E-TPGS which forms micelles in aqueous solution is designed to solubilize fluphenazine HCl without inducing precipitation, and provides administration of the composition to the mammal without the clinical side effects associated with Cremophor.

When fluphenazine-HCl is used to treat a multiple myleoma patient, the following considerations are taken into account. The optimality of multi-bolus and infusion regimens for fluphenazine HCl is dependent on 2-compartment linear PK model calculations, using as constraints, the limited water solubility of fluphenazine, and other factors. Due to large Vₚ lipid-rich peripheral tissue compartment and large Clₜ and Cl_{d} values, a conventional single-bolus loading dose and constant-rate infusion cannot meet the requirement to rapidly achieve central-compartment drug concentrations in the micromolar range on a time-scale commensurate with the rapid up- and down-regulation of 5-HT receptor expression in the myeloma cells and also be expected to sustain such concentrations, with acceptably small deviations from the desired plateau levels. Correspondingly, the conventional parenteral fluphenazine HCl formulation is contained in 10 ml vials at 2.5 mg/ml (25 mg total per vial), such that the requisite bolus dose would entail administration of substantial volume too large to be considered a “bolus” per se to be administered over a few seconds to several minutes. This would more accurately be considered to be an infusion that should be administered over a period of time of not less than 20 minutes. This is a short interval compared to the 16-hour duration of the course of treatment, but is far longer than what is ordinarily termed a “bolus”. This is clinically significant insofar as the target population is frail, predominantly elderly, generally has reduced kidney function either because of age or the presence of myeloma paraprotein, and may be sensitive to abrupt volume loading. Furthermore, there are additional safety considerations related to preventing cardiac and CNS adverse events that suggest using a a moderate rate of infusion rate and a longer time interval (for example, between about 20 to about 40 minutes) for administering a bolus dose of fluphenazine to a patient. During administration, close observation of each patient is required and there must be an immediate capability for discontinuing administration in the event of adverse clinical effects. If conventional, rapid bolus doses of fluphenazine were administered to a patient, it is not possible to prevent adverse clinical effects after the dose has been administered. Mitigation of any such adverse effects may prove difficult because it may not be possible to dialyze away the excess fluphenazine from the patient.

The present invention provides that fluphenazine-HCl is administered to a human multiple myeloma patient, wherein the fluphenazine is formulated with vitamin E derivatives, such as vitamin E-TPGS, or cyclodextrins, such as Captisol™. In one embodiment, the method comprises administering to the patient a pharmaceutical formulation comprising fluphenazine HCl and vitamin E-TPGS or Captisol™ at a dose of 0.1-50 mg/kg of body weight, preferably 1-20 mg/kg, more preferably 1-10 mg/kg, and most preferably 2-8 mg/kg of body weight. The administration may be repeated, preferably every two weeks, and more preferably every three weeks or more. This formulation is preferably administered parenterally to a patient having multiple myeloma or uncontrolled B-cell or plasma cell proliferation.

In addition, fluphenazine HCl formulated with vitamin E-TPGS or Captisol™ may be better tolerated by patient due to lack of hypersensitivity caused by Cremophor, and therefore could be administered in a shorter infusion time more frequently. A long infusion time, such as a 16-hr infusion, requires patients to stay in a hospital and be monitored for the entire period of infusion, thus increasing patients' inconvenience and expenses. Infusion of fluphenazine HCl over a shorter period of time, e.g., 3 hours, would allow out-patient treatment of patients, thereby reducing the cost and discomfort to the patient. Moreover, shorter duration of infusion and lower dosage of fluphenazine HCl may induce less myelosuppression, thereby reducing the incidence of infections and fever episodes. For example, fluphenazine HCl formulated with vitamin E-TPGS or Captisol™ may be administered to a cancer patient by infusion for 3 hours or a shorter time once every week.

Although fluphenazine HCl formulated with vitamin E-TPGS or Captisol™ should not cause hypersensitivity in patients, a desensitizer may optionally be administered to the patients in order to reduce any potential anaphylactic or hypersensitive responses such as allergic reactions, or other reactions. Examples of desensitizers include, but are not limited to, steroids, such as dexamethasone, prednisone and hydrocortisone, antihistamines, such as diphenhydramine, and H-2 receptor blockers, such as cimetidine or ranitidine. The desensitizer or a combination of desensitizers is preferably administered to the patient prior to treatment with fluphenazine HCl formulated with vitamin E-TPGS or Captisol™.

It is also contemplated that the formulations of the present invention are useful for treatment of other diseases with fluphenazine. Such other diseases include, without limitation, psoriasis, and other autoimmune diseases, such as those described in U.S. Patent Application Publication No. 2003/0100570. In addition, the use of fluphenazine and derivatives thereof for modulating the immune response is described in PCT Application No. PCT/US03/19595. Each of these references is incorporated by reference herein in their entirety. Further, treatment of psoriasis using phenothiazine compounds is suggested in U.S. Patent Application Publication Nos. US2004/0029860 and US2005/0013853 to Gil-Ad et al. However, these patent applications do not disclose intralesional delivery of the compound. Rather, the disclosure of these references makes clear that phenothiazine compounds administered for treatment of psoriasis should be administered topically to the patient in the form of salves, gels or ointments, wherein the skin of the patient is not punctured in any way. Alternatively, these references disclose parenteral delivery of phenothiazine compounds to a patient for treatment of psoriasis. Intralesional administration of fluphenazine to a psoriatic patient is disclosed in a U.S. patent application entitled “Intralesional Treatment of Psoriasis” filed simultaneously herewith, which is hereby incorporated by reference herein in its entirety.

Combination Therapy of Fluphenazine HCl with Other Antineoplastic Agents

It is understood that the invention should not be construed to be limited to the administration of fluphenazine alone to a patient having cancer, in particular multiple myeloma. Rather, a wide variety of antineoplastic agents may have a therapeutically additive or synergistic effect with the fluphenazine HCl formulations of the present invention. Such antineoplastic agents may be hyperplastic inhibitory agents that addictively or synergistically combine with the fluphenazine HCl formulation to inhibit undesirable cell growth, such as inappropriate cell growth resulting in undesirable benign conditions or tumor growth. Examples of such antineoplastic agents include, but are not limited to, alkylating agents, antibiotic agents, antimetabolic agents, hormonal agents, plant-derived agents, and biologic agents.

The alkylating agents may be polyfunctional compounds that have the ability to substitute alkyl groups for hydrogen ions. Non-limiting examples of alkylating agents include bischloroethylamines (nitrogen mustards, e.g. melphalan, chlorambucil, cyclophosphamide, ifosfamide, mechlorethamine, uracil mustard), aziridines (e.g. thiotepa), alkyl alkone sulfonates (e.g. busulfan), nitrosoureas (e.g. carmustine, lomustine, streptozocin), nonclassic alkylating agents (altretamine, dacarbazine, and procarbazine), platinum compounds (carboplastin and cisplatin). These compounds react with phosphate, amino, hydroxyl, sulfihydryl, carboxyl, and imidazole groups. Under physiological conditions, these compounds ionize to produce positively charged ions that attach to susceptible nucleic acids and proteins, leading to cell cycle arrest and/or cell death. Combination therapy including the fluphenazine HCl formulation of the invention and an alkylating agent is therefore contemplated to be included in the present invention.

Examples of antibiotic agents include, but are not limited to, anthracyclines (e.g. doxorubicin, daunorubicin, epirubicin, idarubicin and anthracenedione), mitomycin C, bleomycin, dactinomycin, plicatomycin. These antibiotic agents interfere with cell growth by targeting different cellular components. For example, anthracyclines are generally believed to interfere with the action of DNA topoisomerase II in the regions of transcriptionally active DNA, which leads to DNA strand scissions. Bleomycin is generally believed to chelate iron and forms an activated complex, which then binds to the bases in DNA causing strand scissions and cell death. Combination therapy including the fluphenazine HCl formulation of the invention and an antibiotic agent is also contemplated as being included in the present invention.

The antimetabolic agents are a group of drugs that interfere with metabolic processes vital to the physiology and proliferation of cancer cells. Actively proliferating cancer cells require continuous synthesis of large quantities of nucleic acids, proteins, lipids, and other vital cellular constituents. Many antimetabolic agents inhibit the synthesis of purine or pyrimidine nucleosides or inhibit the enzymes involved in DNA replication. Some antimetabolites also interfere with the synthesis of ribonucleosides and RNA and/or amino acid metabolism and protein synthesis as well. By interfering with the synthesis of vital cellular constituents, antimetabolic agents can delay or arrest the growth of cancer cells. Examples of antimetabolic agents include, but are not limited to, bortezomib, thalidomide, arsenic trioxide, fluorouracil (5-FU), floxuridine (5-FUdR), methotrexate, leucovorin, hydroxyurea, thioguanine (6-TG), mercaptopurine (6-MP), cytarabine, pentostatin, fludarabine phosphate, cladribine (2-CDA), asparaginase, and gemcitabine. Combination therapy including the fluphenazine HCl formulation of the invention and an antimetabolic agent is also contemplated as being included in the invention.

Hormonal agents are a group of drug that regulate the function, growth or development of their target organs. Many hormonal agents are steroids and their derivatives and analogs thereof, such as estrogens, androgens, and progestins. These hormonal agents may serve as antagonists of receptors for sex steroids to down regulate receptor expression and transcription of vital genes. Non-limiting examples of such hormonal agents include synthetic estrogens (e.g. diethylstibestrol), antiestrogens (e.g. tamoxifen, toremifene, fluoxymesterol and raloxifene), antiandrogens (bicalutamide, nilutamide, flutamide), aromatase inhibitors (e.g., aminoglutethimide, anastrozole and tetrazole), ketoconazole, goserelin acetate, leuprolide, megestrol acetate and mifepristone. Combination therapy including the fluphenazine HCl formulation of the invention and a hormonal agent is also included in the present invention.

Non-limiting examples of plant-derived agents include vinca alkaloids (e.g., vincristine, vinblastine, vindesine, vinzolidine and vinorelbine), podophyllotoxins (e.g., etoposide (VP-16) and teniposide (VM-26)), camptothecin (e.g., 20(S)-camptothecin, 9-nitro-20(S)-camptothecin and 9-amino-20(S)-camptothecin). These plant-derived agents generally act as antimitotic agents that bind to tubulin and inhibit mitosis. Podophyllotoxins such as etoposide are believed to interfere with DNA synthesis by interacting with topoisomerase II, leading to DNA strand scission. Combination therapy including the fluphenazine HCl formulation of the invention and a plant-derived agent is also included in the present invention.

Biologic agents are a group of biomolecules that elicit cancer/tumor regression when used alone or in combination with chemotherapy and/or radiotherapy. Examples of biologic agents include, but are not limited to, immunomodulating proteins such as cytokines, monoclonal antibodies directed against tumor antigens, tumor suppressor genes, and cancer vaccines. Combination therapy including the fluphenazine HCl formulation of the invention an a biologic agent is included in the present invention.

Cytokines possess profound immunomodulatory activity. Some cytokines such as interleukin-2 (IL-2, aldesleukin) and interferon α (IFN-α) demonstrate antitumor activity and have been approved for the treatment of patients with metastatic renal cell carcinoma and metastatic malignant melanoma. IL-2 is a T-cell growth factor that is central to T-cell-mediated immune responses. The selective antitumor effects of IL-2 on some patients are believed to be the result of a cell-mediated immune response that discriminate between self and nonself. Examples of interleukins that may be used in conjunction with the inventive fluphenazine HCl formulation include, but are not limited to, interleukin 2 (IL-2), and interleukin 4 (IL-4), interleukin 12 (IL-12).

Interferon-α (IFN-α) is the name given to a group of compounds that includes more than twenty three related subtypes with overlapping activities. All of the IFN-α subtypes within the scope of the present invention. IFN-α has demonstrated activity against many solid and hematologic malignancies, the latter appearing to be particularly sensitive. Additional interferons include interferon β and interferon γ. Thus, examples of interferons that may be used in conjunction with fluphenazine HCl formulated with vitamin E-TPGS or Captisol™ include, but are not limited to, interferon α, interferon β, and interferon γ.

Other cytokines that may be used in conjunction with fluphenazine HCl formulated with vitamin E-TPGS or Captisol™ include those cytokines that exert profound effects on hematopoiesis and immune functions. Examples of such cytokines include, but are not limited to erythropoietin (epoietin α), granulocyte-CSF (filgrastim) and granulocyte/macrophage-CSF (sargramostim). These cytokines may be used in conjunction with fluphenazine to reduce chemotherapy-induced myelotoxicity.

Other immuno-modulating agents other than cytokines may also be used in conjunction with fluphenazine to inhibit abnormal cell growth. Examples of such immuno-modulating agents include, but are not limited to bacillus Calmette-Guerin, levamisole, and octreotide.

Monoclonal antibodies directed against tumor antigens are also contemplated. For example, the monoclonal antibody, trastruzumab, is specific for human epidermal growth factor receptor-2 (HER2) that is overexpressed in some breast tumors including metastatic breast cancer. Therapeutic regimens including parenteral fluphenazine HCl formulations that are the subject of the present invention used concomitantly with monoclonal antibodies may have synergistic effects on cancer and reduce sides affects associated with these chemotherapeutic agents and are therefore included in the invention.

The preferred types of cancers or malignant tumors that can be treated with the formulations of the invention multiple myeloma, Burkitt's lymphoma, and other B-cell lymphomas.

The invention is now described with reference to the following Examples. These Examples are provided for the purpose of illustration only and the invention should in no way be construed as being limited to these Examples, but rather should be construed to encompass any and all variations which become evident as a result of the teaching provided herein.

### EXPERIMENTAL EXAMPLES

### Example 1

Fluphenazine HCl (10 mg) was dissolved in ethanol. Vitamin E TPGS (VTPGS, 700 mg, Eastman Chemical Company) was melted at 50° C. and dissolved separately in ethanol in a ratio of 3:1, respectively. Fluphenazine HCl and VTPGS solutions were mixed and ethanol was added to the solution to a final amount of 300 mg, resulting in a 7:3 weight ratio of VTPGS to ethanol. Anhydrous ascorbic acid (5 mg) was then added to the mixture. The resultant stock solution (ICI-02-A) appeared clear and yellow in color. The total volume of the stock solution was 25 ml.

Aliquots of the stock solution (ICI-02-A) were transferred into vials at 5 ml/vial, and incubated at 4° C., 25° C., 40° C. and 50° C., respectively, for the periods of time shown in Tables 1A, 1B, 1C and 1D. Samples were taken at one week or predetermined intervals and tested for chemical stability. The stability testing was performed using HPLC. A LC-F (penta-fluorophenyl bonded phase) 5 μm, 100 Å pore size, 4.6×250 mm column. A UV detector set at 227 nm was used. The mobile phase was made up of a 37:58:5 mixture of ACN:Water:MeOH (containing 1 ml/L of H₃PO₄). The flow rate was 1.2 ml/minute. The diluent used was acidic methanol (MeOH containing 0.1% acetic acid). The sample concentration was 0.01 mg/ml. The injection volume was 20 μl. The retention time was 14.5 minutes. The results are presented in Tables 1A, 1B, 1C and 1D.

One milliliter of the stock solution (ICI-02-A) was diluted to 5.0 ml with 0.9% NaCl and observed for precipitation at room temperature for a period of at least 24 hrs. The diluted solution had a pH of about 4. The formulation did not exhibit any signs of precipitation after 24 hrs,

### Example 2

Fluphenazine HCl (10 mg) was dissolved in ethanol. Vitamin E TPGS (VTPGS, 600 mg) was melted at 50° C. and dissolved separately in ethanol in a ratio of 3:1, respectively. The fluphenazine HCl and VTPGS solutions were mixed and ethanol was added to the solution to a final amount of 400 mg, resulting in a 6:4 weight ratio of VTPGS to ethanol. Anhydrous ascorbic acid (5 mg) was then added to the mixture. The resultant stock solution (ICI-02-B) appeared clear and yellow in color. The total volume of the stock solution was 25 ml.

Aliquots of the stock solution (ICI-02-B) were transferred into vials at 5 m/vial, and incubated at 4° C., 25° C., 40° C. and 50° C., respectively, for the periods of time shown in Tables 1A, 1B, 1C and 1D. Samples were taken at one week or predetermined intervals and were tested for chemical stability of fluphenazine HCl. The stability testing was performed using the method outlined in Example 1. The results are shown in Tables 1A, 1B, 1C and 1D. One milliliter of the stock solution (ICI-02-B) was diluted to 5.0 ml with 0.9% NaCl and observed for precipitation at room temperature for a period of at least 24 hrs. The diluted solution had a pH of about 4. The formulation did not exhibit any signs of precipitation after over 24 hrs or more.

### Example 3

Fluphenazine HCl (10 mg) was dissolved in ethanol. Vitamin E TPGS (VTPGS, 500 mg) was melted at 50° C. and dissolved separately in ethanol in a ratio of 3:1, respectively. The fluphenazine HCl and VTPGS solutions were mixed and ethanol was added to the solution to a final amount of 500 mg, resulting in a 5:5 weight ratio of VTPGS to ethanol. Anhydrous ascorbic acid (5 mg) was then added to the mixture. The resultant stock solution (ICI-02-C) appeared clear and yellow in color. The total volume of the stock solution was 25 ml.

Aliquots of the stock solution (ICI-02-C) were transferred into vials at 5 m/vial, and incubated at 4° C., 25° C., 40° C. and 50° C., respectively, for periods of time as listed in Tables 1A, 1B, 1C and 1D. Samples were taken at one week intervals and tested for chemical stability of fluphenazine HCl. The stability testing was performed using the method outlined in Example 1. The results are shown in Tables 1A, 1B, 1C and 1D.

One milliliter of the stock solution (ICI-02-C) was diluted to 5.0 ml with 0.9% NaCl and observed for precipitation at room temperature for a period of at least 24 hrs. The diluted solution had a pH of about 4. The formulation did not exhibit any signs of precipitation over 24 hrs.

TABLE 1A

| 4° C. | % Fluphenazine HCl Recovery | | |
|---|---|---|---|
| Time (week) | ICI-02-A | ICI-02-B | ICI-02-C |
| 0 | 100 | 100 | 100 |
| 1 | 99 | 102 | 98 |
| 2 | 98 | 98 | 98 |
| 3 | 99 | 98 | 102 |

TABLE 1B

| 25° C. | % Fluphenazine HCl Recovery | | |
|---|---|---|---|
| Time (week) | ICI-02-A | ICI-02-B | ICI-02-C |
| 0 | 100 | 100 | 100 |
| 1 | 99 | 99 | 101 |
| 2 | 98 | 99 | 98 |
| 3 | 101 | 102 | 100 |

TABLE 1C

| 40° C. | % Fluphenazine HCl Recovery | | |
|---|---|---|---|
| Time (week) | ICI-02-A | ICI-02-B | ICI-02-C |
| 0 | 100 | 100 | 100 |
| 1 | 100 | 101 | 100 |
| 2 | 97 | 98 | 101 |
| 3 | 99 | 101 | 102 |

TABLE 1D

| 50° C. | % Fluphenazine HCl Recovery | | |
|---|---|---|---|
| Time (week) | ICI-02-A | ICI-02-B | ICI-02-C |
| 0 | 100 | 100 | 100 |
| 1 | 98 | 99 | 99 |
| 2 | 98 | 99 | 98 |
| 3 | 97 | 98 | 98 |

### Example 4

Chemical and physical stability of the fluphenazine HCl formulation following dilution with normal saline was determined at certain time points after dilution. Table 2 lists percentages of fluphenazine HCl at indicated time points for a period of 24 hrs after 1:10 dilution of two fluphenazine HCl formulations: fluphenazine HCl at 10 mg/g in 50:50 ethanol: vitamin E TPGS, and fluphenazine HCl at 12.5 mg/g in 50:50 ethanol:vitamin E TPGS.

TABLE 2

| | |
|---|---|
| Time (h) | % Fluphenazine Recovery |

Fluphenazine HCl (10.0 mg/g) at 1:10 dilution (1.00 mg/g)

| | |
|---|---|
| 0 | 99.76 |
| 2 | 99.74 |
| 4 | 99.55 |
| 8 | 99.44 |
| 24 | 99.15 |

Fluphenazine HCl (12.5 mg/g) at 1:10 dilution (1.25 mg/g)

| | |
|---|---|
| 0 | 99.96 |
| 2 | 99.81 |
| 4 | 99.61 |
| 8 | 99.43 |
| 24 | 99.25 |

Table 3 lists observation of precipitation at indicated time points after dilution of the fluphenazine HCl formulation according the present invention with normal saline at indicated ratios. The fluphenazine HCl formulation has fluphenazine HCl at 12.5 mg/g in 50:50 ethanol: vitamin E TPGS.

TABLE 3

| | Precipitation | | | | |
|---|---|---|---|---|---|
| Dilution Ratio | 0 h | 24 h | 36 h | 48 h | 72 h |
| 1:5 | None | None | Yes | Yes | Yes |
| 1:6 | None | None | Yes | Yes | Yes |
| 1:7 | None | None | None | Yes | Yes |
| 1:8 | None | None | None | None | Yes |
| 1:9 | None | None | None | None | None |
| 1:10 | None | None | None | None | None |

### Example 5

Fluphenazine HCl (30 mg) was dissolved in ethanol. Captisol™ was dissolved separately in water at 40% w/v. The fluphenazine HCl and Captisol™ solutions were mixed to a final amount of 30 mg/g fluphenazine HCl: Captisol™. Anhydrous ascorbic acid (5 mg) was then added to the mixture. The resultant stock solution (ICI-02-D) appeared clear and yellow in color. The total volume of the stock solution was 25 ml.

Aliquots of the stock solution (ICI-02-D) were transferred into vials at 5 ml/vial, and incubated at 4° C., 25° C., 40° C. and 50° C., respectively, for periods of time as listed in Tables 4A, 4B, 4C and 4D. Samples were taken at one week or predetermined intervals and tested for chemical stability. The stability testing was performed using HPLC. A LC-F (penta-fluorophenyl bonded phase) 5 μm, 100 Å pore size, 4.6×250 mm column was used. A UV detector set at 227 nm was used. The mobile phase was made up of a 37:58:5 mixture of ACN:Water:MeOH (containing 1 m/L of H₃PO₄). The flow rate was 1.2 ml/minute. The diluent used was acidic methanol (MeOH containing 0.1% acetic acid). The sample concentration was 0.01 mg/ml. The injection volume was 20 μl. The retention time was 14.5 minutes. The results are shown in Tables 4A, 4B, 4C and 4D.

One milliliter of the stock solution (ICI-02-D) was diluted to 5.0 ml with 0.9% NaCl and observed for precipitation at room temperature for a period of at least 24 hrs. The diluted solution had a pH of about 6. The formulation did not exhibit any signs of precipitation after over 24 hrs.

### Example 6

Fluphenazine HCl (40 mg) was dissolved in ethanol. Captisol™ was dissolved separately in water at 40% w/v. The fluphenazine HCl and Captisol™ solutions were mixed to a final amount of 40 mg/g fluphenazine HCl: Captisol™. Anhydrous ascorbic acid (5 mg) was then added to the mixture. The resultant stock solution (ICI-02-E) appeared clear and yellow in color.

Aliquots of the stock solution (ICI-02-E) were transferred into vials at 5 ml/vial, and incubated at 4° C., 25° C., 40° C. and 50° C., respectively, for periods of time as listed in Tables 4A, 4B, 4C and 4D. Samples were taken at one week or predetermined intervals and tested for chemical stability of fluphenazine HCl. The stability testing was performed using the method outlined in Example 1. The results are shown in Tables 4A, 4B, 4C and 4D. One milliliter of the stock solution (ICI-02-E) was diluted to 5.0 ml with 0.9% NaCl and observed for precipitation at room temperature for a period of at least 24 hrs. The diluted solution had a pH of about 6. The formulation did not exhibit any signs of precipitation after over 24 hr or greater.

### Example 7

Fluphenazine HCl (50 mg) was dissolved in ethanol. Captisol™ was dissolved separately in water at 40% w/v. The fluphenazine HCl and Captisol™ solutions were mixed to a final amount of 50 mg/g fluphenazine HCl: Captisol™. Anhydrous ascorbic acid (5 mg) was then added to the mixture. The resultant stock solution (ICI-02-F) appeared clear and yellow in color.

Aliquots of the stock solution (ICI-02-F) were transferred into vials at 5 ml/vial, and incubated at 4° C., 25° C., 40° C. and 50° C., respectively, for periods of time as listed in Tables 4A, 4B, 4C and 4D. Samples were taken at one week intervals and tested for chemical stability of fluphenazine HCl. The stability testing was performed using the method outlined in Example 1. The results are shown in Tables 4A, 4B, 4C and 4D.

One milliliter of the stock solution (ICI-02-F) was diluted to 5.0 ml with 0.9% NaCl and observed for precipitation at room temperature for a period of at least 24 hrs. The diluted solution had a pH of about 6. The formulation did not exhibit any signs of precipitation after over 24 hrs.

TABLE 4A

| 4° C. | % Fluphenazine HCl Recovery | | |
|---|---|---|---|
| Time (week) | ICI-02-D | ICI-02-E | ICI-02-F |
| 0 | 100 | 100 | 101 |
| 1 | 100 | 99 | 99 |
| 2 | 100 | 99 | 99 |
| 3 | 99 | 98 | 97 |

TABLE 4B

| 25° C. | % Fluphenazine HCl Recovery | | |
|---|---|---|---|
| Time (week) | ICI-02-D | ICI-02-E | ICI-02-F |
| 0 | 100 | 100 | 101 |
| 1 | 99 | 98 | 101 |
| 2 | 99 | 99 | 98 |
| 3 | 99 | 102 | 98 |

TABLE 4C

| 40° C. | % Fluphenazine HCl Recovery | | |
|---|---|---|---|
| Time (week) | ICI-02-D | ICI-02-E | ICI-02-F |
| 0 | 100 | 100 | 100 |
| 1 | 99 | 100 | 101 |
| 2 | 97 | 96 | 100 |
| 3 | 99 | 97 | 99 |

TABLE 4D

| 50° C. | % Fluphenazine HCl Recovery | | |
|---|---|---|---|
| Time (week) | ICI-02-D | ICI-02-E | ICI-02-F |
| 0 | 102 | 100 | 101 |
| 1 | 99 | 101 | 102 |
| 2 | 98 | 99 | 97 |
| 3 | 96 | 97 | 99 |

### Example 8

Chemical and physical stability of the CyDex Captisol™ fluphenazine HCl formation following dilution with normal saline was determined at certain time points after the dilution. Table 5 lists percentages of fluphenazine HCl at indicated time points for a period of 24 hrs after 1:10 dilution of two fluphenazine HCl formulations: fluphenazine HCl at 60 mg/g in Captisol™, and fluphenazine HCl at 75 mg/g in Captisol™.

TABLE 5

| | |
|---|---|
| Time (h) | % Fluphenazine Recovery |

Fluphenazine HCl (60 mg/g) at 1:10 dilution (6.0 mg/g)

| | |
|---|---|
| 0 | 99.84 |
| 2 | 99.77 |
| 4 | 99.62 |
| 8 | 99.51 |
| 24 | 99.35 |

Fluphenazine HCl (75 mg/g) at 1:10 dilution (7.5 mg/g)

| | |
|---|---|
| 0 | 99.90 |
| 2 | 99.86 |
| 4 | 99.65 |
| 8 | 99.53 |
| 24 | 99.44 |

Table 6 lists observation of precipitation at indicated time points after dilution of the fluphenazine HCl formulation according the present invention with normal saline at indicated ratios. The fluphenazine HCl formulation comprises fluphenazine HCl at 75 mg/g in Captisol™.

TABLE 6

| | Precipitation | | | | |
|---|---|---|---|---|---|
| Dilution Ratio | 0 h | 24 h | 36 h | 48 h | 72 h |
| 1:5 | None | None | None | None | None |
| 1:6 | None | None | None | None | None |
| 1:7 | None | None | None | None | None |
| 1:8 | None | None | None | None | None |
| 1:9 | None | None | None | None | None |
| 1:10 | None | None | None | None | None |

The disclosures of each and every patent, patent application, and publication cited herein are hereby incorporated herein by reference in their entirety.

While the invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A pharmaceutical composition for treating a patient in need thereof, said composition comprising:
a water-miscible, non-aqueous fluphenazine HCl formulation comprising fluphenazine HCl and ascorbic acid dissolved in a water-miscible non-aqueous solvent;
a pharmaceutically-acceptable, water-miscible solubilizer, wherein said solubilizer is selected from the group consisting of solubilizers having the general formula:
R₁ COOR₂, R₁ CONR₂, and R₁ COR₂,
wherein R1 is a derivative of d-α-tocopherol and R₂ is a hydrophilic moiety.

2. The pharmaceutical composition of claim 1, wherein said solubilizer is d-α-tocopherol polyethylene glycol succinate.

3. The pharmaceutical composition of claim 2, wherein said d-α-tocopherol polyethylene glycol succinate is d-α-tocopherol polyethylene glycol 1000 succinate.

4. The pharmaceutical composition of claim 1, wherein said water-miscible non-aqueous solvent is an alcohol.

5. The pharmaceutical composition of claim 4, wherein said solvent is selected from the group consisting of ethanol, propylene glycol, benzyl alcohol, and polyethylene glycol (PEG).

6. The pharmaceutical composition of claim 1, wherein said solvent is an amide.

7. The pharmaceutical composition of claim 6, wherein said solvent is selected from the group consisting of 2-pyrrolidone, N-methyl-pyrrolidone and N,N-dimethyl acetamide.

8. The pharmaceutical composition of claim 2, wherein the weight ratio of said d-α-tocopherol polyethylene glycol succinate to said solvent is between about 90:10 and about 40:60.

9. The pharmaceutical composition of claim 8, wherein the weight ratio of said d-α-tocopherol polyethylene glycol succinate to said solvent is between about 70:30 and about 45:55.

10. The pharmaceutical composition of claim 9, wherein the weight ratio of said d-α-tocopherol polyethylene glycol succinate to said solvent is about 50:50.

11. The pharmaceutical composition of claim 1, wherein said solubilizer is a β-cyclodextrin sulfobutyl ether.

12. The pharmaceutical composition of claim 11, wherein the weight ratio of said β-cyclodextrin sulfobutyl ether to said solvent is between about 50:50 and about 30:70.

13. The pharmaceutical composition of claim 1, wherein said water-miscible, non-aqueous fluphenazine HCl formulation is diluted with infusion fluid to form a pharmaceutically acceptable aqueous solution.

14. The pharmaceutical composition of claim 13, wherein the amount of ascorbic acid in said fluphenazine HCl formulation before said dilution is between about 0.1 to about 5% w/w.

15. The pharmaceutical composition of claim 14, wherein the amount of ascorbic acid in said fluphenazine HCl formulation before said dilution is between about 0.4 to about 2% w/w.

16. The pharmaceutical composition of claim 15, wherein the amount of ascorbic acid in said fluphenazine HCl formulation before said dilution is between about 0.5 to about 1% w/w.

17. A method of treating a disease associated with undesirable plasma cell proliferation in a patient, said method comprising
providing a water-miscible, non-aqueous fluphenazine HCl formulation comprising fluphenazine HCl and ascorbic acid dissolved in a water-miscible non-aqueous solvent and a pharmaceutically-acceptable, water-miscible solubilizer, wherein said solubilizer is selected from the group consisting of solubilizers having the general formula:
R₁ COOR₂, R₁ CONR₂, and R₁ COR₂,
wherein R1 is a derivative of d-α-tocopherol and R₂ is a hydrophilic moiety;
diluting said water-miscible, non-aqueous fluphenazine HCl formulation into a pharmaceutically acceptable aqueous solution to form a pharmaceutical composition; and
parenterally administering to the patient said pharmaceutical composition at a dose of 0.1 to 20 mg/kg body weight, thereby treating said disease.

18. The method of claim 17, wherein said pharmaceutical composition is administered intravenously to said patient.

19. The method of claim 17, wherein said fluphenazine HCl is administered to said patient at a dose of about 0.5 to about 5 mg/kg of body weight.

20. The method of claim 19, wherein said fluphenazine HCl is administered to said patient at a dose of about 1 to about 10 mg/kg of body weight.

21. The method of claim 20, wherein said fluphenazine HCl is administered to said patient at a dose of about 2 to about 8 mg/kg of body weight.

22. The method of claim 21, wherein said fluphenazine HCl is administered to said patient at a dose of about 3 to about 6 mg/kg of body weight.

23. The method of claim 17, wherein the said fluphenazine HCl formulation is diluted with infusion fluid.

24. The method of claim 23, wherein the amount of ascorbic acid in said fluphenazine HCl formulation before said dilution is between about 0.1 to about 5% w/w.

25. The method of claim 24, wherein the amount of ascorbic acid in said fluphenazine HCl formulation before said dilution is between about 0.4 to about 2% w/w.

26. The method of claim 25, wherein the amount of ascorbic acid in said fluphenazine HCl formulation before said dilution is between about 0.5 to about 1% w/w.

27. The method of claim 23, wherein said pharmaceutical composition is administered to said patient by infusion for less than 18 hours.

28. The method of claim 27, wherein said pharmaceutical composition is administered to said patient by infusion for less than 3 hours.

29. The method of claim 17, wherein the said pharmaceutical composition is administered to said patient once every week.

30. The method of claim 17, wherein said pharmaceutical composition is administered to said patient once every two weeks.

31. The method of claim 17, wherein said pharmaceutical composition is administered to said patient once every three weeks.

32. The method of claim 17, wherein said solubilizer is d-α-tocopherol polyethylene glycol succinate.

33. The method of claim 32, wherein said d-α-tocopherol polyethylene glycol succinate is d-α-tocopherol polyethylene glycol 1000 succinate.

34. The method of claim 17, wherein said water-miscible non-aqueous solvent is an alcohol.

35. The method of claim 17, wherein said solvent is selected from the group consisting of ethanol, propylene glycol, benzyl alcohol, and polyethylene glycol (PEG).

36. The method of claim 17, wherein said solvent is an amide.

37. The method of claim 36, wherein said solvent is selected from the group consisting of 2-pyrrolidone, N-methyl-pyrrolidone and N,N-dimethyl acetamide.

38. The method of claim 37, wherein the weight ratio of said d-α-tocopherol polyethylene glycol succinate to said solvent is between about 90:10 and about 40:60.

39. The method of claim 38, wherein the weight ratio of said d-α-tocopherol polyethylene glycol succinate to said solvent is between about 70:30 and about 45:55.

40. The method of claim 39, wherein the weight ratio of said d-α-tocopherol polyethylene glycol succinate to said solvent is about 50:50.

41. The method of claim 17, wherein said solubilizer is a β-cyclodextrin sulfobutyl ether.

42. The method of claim 41, wherein the weight ratio of said α-cyclodextrin sulfobutyl ether to said solvent is between about 50:50 and about 30:70.
